(19) United States
(12) Patent Application Publication
(10) Pub. No.: US 2026/0335241 A1
(43) Pub. Date: Oct. 29, 2026

(54) PHARMACEUTICAL COMPOSITIONS COMPRISING 5-ETHYL-4-METHYL- N-[4-[(2 S) MORPHOLIN-2-YL]PHENYL]-1H-PYRAZOLE-3-CARBOXAMIDE

(71) Applicant: Hoffmann-La Roche Inc., Little Falls, NJ (US)

(72) Inventors: Davide DE FRANCO, Liestal (CH); Andreas EWERT, Buggingen (DE); Emmanuela GAVI, Basel (CH); Reto MAURER, Basel (CH)

(73) Assignee: Hoffmann-La Roche Inc., Little Falls, NJ (US)

(21) Appl. No.: 19/103,543

(22) PCT Filed: Aug. 10, 2023
(86) PCT No.: PCT/EP2023/072132

§ 371 Date: Feb. 12, 2025

(30) Foreign Application Priority Data
Aug. 12, 2022 (EP) 22190194.5

(51) Int. Cl. A61K 9/20; A61K 9/28; A61K 31/5377

(52) U.S. Cl. CPC A61K 9/2054; A61K 9/2018; A61K 9/2027; A61K 9/2095; A61K 9/2813; A61K 9/2826; A61K 9/2853; A61K 9/2866; A61K 31/5377

(57) ABSTRACT

The present invention relates to pharmaceutical compositions comprising 5-ethyl-4-methyl-N-[4-[(2S) morpholin-2-yl]phenyl]-1H-pyrazole-3-carboxamide (ralmitaront), to processes for their preparation and their use in medical treatment.

### FIELD OF THE INVENTION

The present invention relates to pharmaceutical compositions comprising 5-ethyl-4-methyl-N-[4-[(2S) morpholin-2-yl]phenyl]-1H-pyrazole-3-carboxamide (Formula I), to processes for their preparation and their use in medical treatment.

### BACKGROUND OF THE INVENTION

WO2017157873, the entire contents of which are incorporated herein by reference, discloses the TAAR1 agonist 5-ethyl-4-methyl-N-[4-[(2S) morpholin-2-yl]phenyl]-1H-pyrazole-3-carboxamide (Formula I), which is useful for the treatment of certain diseases and disorders of the central nervous system.

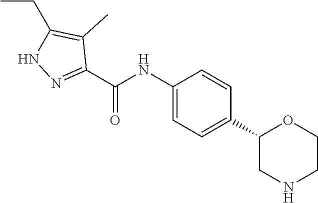 [SMILES: CCc1[nH]nc(C(=O)Nc2ccc([C@H]3CNCCO3)cc2)c1C]

The compound of Formula I is also known under the INN ralmitaront (WHO Drug Information, Vol. 33, No. 2, 2019, p. 323).

For some of the medical indications of ralmitaront, such as schizophrenia, small sized tablets are required to improve patient compliance. Small sized tablets require the provision of API-excipient blends with a high drug load, for example, a drug load of 30% wt/wt API. WO2017157873 discloses tablet and capsule formulations with high loads of ralmitaront. However, it was found that ralmitaront is cohesive and API-excipient blends containing it, especially in high amounts, do not flow well, making tablet production challenging. Additionally, the compound includes a secondary amine and a manufacturing process under high humidity conditions, such as wet granulation disclosed in WO2017157873 would increase the risk of nitrosamine formation.

Furthermore, it would be desirable to manufacture ralmitaront tablets in a continuous fashion, rather than in a conventional batch setup. Continuous manufacturing has commercial manufacturing advantages, such as improved process control, reduced product handling, and real time release efficiencies. The overall result is a more robust, controllable, and scalable process that requires fewer process checks. However, it was found that the blends disclosed in WO2017157873 are not well suited for tablet production in a continuous manufacturing setup.

In summary, there is a high unmet need for new formulations comprising ralmitaront.

### SUMMARY OF THE INVENTION

The present invention provides new formulation blends comprising a high load of ralmitaront that avoid the wet granulation described in WO2017157873. Some of the new blends are suitable for roller compaction, while others are suitable for continuous direct compression, in particular for continuous mini-batch direct compression. The present invention also provides new processes for manufacturing tablets comprising ralmitaront, as well as the use of said tablets in medical therapy.

### BRIEF DESCRIPTION OF THE FIGURES

FIG. 1 depicts a flow chart of the continuous mini-batch direct compression process according to the invention described in Example 1.

FIG. 2 depicts a flow chart of the roller compaction according to the invention described in Example 2.

FIG. 3 depicts the effect of the formulations represented by examples 5, 6 and 7, on the tablet tensile strength and Srel % (standard relative deviation of tablet weight) during the compaction process in the rotary press. “Batch 1” refers to the formulation of Example 5, “batch 2” refers to the formulation of Example 6 and “batch 3” refers to the formulation of Example 7. The tag that accompanies the batch number indicates on what tablet sample the measurement of the variables was made: “Start” means the tablet sample was collected at the start of tableting, “Middle” means the tablet sample was collected during the middle of tableting, “End” means the tablet sample was collected during the end of tableting, “Mixed” the tablet sample is constituted by a mixture of tablets in the middle and end of tableting.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

As used herein, the term “dose strength” relates to the absolute amount of the compound of Formula I in its free base form contained in a tablet formulation according to the invention, expressed in milligrams (mg). Consequently, if the compound of formula I is used in the form of a pharmaceutically acceptable salt, the term “dose strength” relates to the respective free base equivalent.

As used herein, the term “ralmitaront” relates to 5-ethyl-4-methyl-N-[4-[(2S) morpholin-2-yl]phenyl]-1H-pyrazole-3-carboxamide (Formula I), and pharmaceutically acceptable salts thereof, in particular the mono hydrochloric acid salt.

As used herein, the term “filler” refers to a substance added to a pharmaceutical composition to increase the weight and/or size of the pharmaceutical composition. Pharmaceutically acceptable fillers are described in Remington's Pharmaceutical Sciences and listed in Handbook of Pharmaceutical Excipients, Sheskey et al., 2017. Non-limiting examples of fillers are starch (e.g., pregelatinized starch), cellulose (e.g., microcrystalline cellulose) and lactose (e.g., lactose monohydrate). Preferred, yet non-limiting examples of fillers are cellulose and lactose.

As used herein, the term “disintegrant” refers to a substance added to a pharmaceutical composition to help break apart (disintegrate), e.g., after administration, and release the active ingredient, such as Form B described herein. Pharmaceutically acceptable disintegrants are described in Remington's Pharmaceutical Sciences and listed in Handbook of Pharmaceutical Excipients, Sheskey et al., 2017. Non-limiting examples of disintegrants are low substituted hydroxypropyl cellulose and croscarmellose sodium. A preferred, yet non-limiting example of a disintegrant is croscarmellose sodium.

The terms “glidant” and “lubricant” are used herein interchangeably and refer to a substance added to a pharmaceutical composition to help reduce the adherence of a granule of powder to equipment surfaces. Pharmaceutically acceptable glidants are described in Remington's Pharmaceutical Sciences and listed in Handbook of Pharmaceutical Excipients, Sheskey et al., 2017. Non-limiting examples of glidants are sodium stearyl fumarate and magnesium stearate. A preferred, yet non-limiting example of a glidant is sodium stearyl fumarate.

As used herein, the term “flow agent” refers to a substance added to a pharmaceutical composition to enhance product flow by reducing interparticulate friction. Pharmaceutically acceptable flow agents are described in Remington's Pharmaceutical Sciences and listed in Handbook of Pharmaceutical Excipients, Sheskey et al., 2017. Non-limiting examples of flow agents include silicon dioxide (colloidal), polyethylene glycol PEG 6000, fumed silicon dioxide Aerosil® 200, talc and the like. A preferred, yet non-limiting example is silica, colloidal anhydrous.

The term “MicroceLac®” refers to an excipient comprising 75% alpha-lactose monohydrate and 25% microcrystalline cellulose, wherein said alpha-lactose monohydrate and microcrystalline cellulose have been co-processed by spray drying.

The term “SMCC90” refers to an excipient comprising 98% microcrystalline cellulose and 2% colloidal silicon dioxide, wherein said microcrystalline cellulose and colloidal silicon dioxide have been co-processed by spray drying.

The term “Ludipress®” refers to an excipient comprising 93% lactose monohydrate, 3.5% povidone having a K-value of 30 (“Kollidon® 30”) and 3.5% crospovidone having a bulk density of 0.30-0.40 g/mL (“Kollidon® CL”).

The term “HPMC” refers to hydroxypropylmethylcellulose.

As used herein, the term “treating” means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself.

As used herein, the term “preventing” includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

As used herein, the term “patient” refers to a human.

### Novel Tablet Formulation of Ralmitaront

In a first aspect, the present invention provides a tablet comprising:
(i) a kernel; and
(ii) a coating;
wherein the kernel comprises the active ingredient 5-ethyl-4-methyl-N-[4-[(2S) morpholin-2-yl]phenyl]-1H-pyrazole-3-carboxamide (Formula I), or a pharmaceutically acceptable salt thereof,

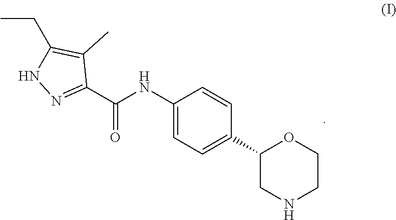 [SMILES: CCc1[nH]nc(C(=O)Nc2ccc([C@H]3CNCCO3)cc2)c1C]

In one embodiment, the tablet according to the invention comprises the compound of Formula I in the form of its mono hydrochloric acid salt.

In one embodiment, the dose strength of the tablet according to the invention is 40 mg to 160 mg, preferably 45 mg to 150 mg, more preferably 45 mg or 150 mg.

In a preferred embodiment, the dose strength of the tablet according to the invention is 45 mg.

In a preferred embodiment, the dose strength of the tablet according to the invention is 150 mg.

In one embodiment, the coating of the tablet according to the invention comprises:
(i) HPMC;
(ii) lactose monohydrate;
(iii) titanium dioxide; and
(iv) macrogol.

In one embodiment:
(i) HPMC represents 34%+1% of the total weight of the coating;
(ii) lactose monohydrate represents 28%+1% of the total weight of the coating;
(iii) titanium dioxide represents 26%+1% of the total weight of the coating; and
(iv) macrogol represents 12%+1% of the total weight of the coating.

In one embodiment, the coating of the tablet according to the invention is Opadry II White.

### Formulation for Continuous Mini-Batch Direct Compression

### Formulation A

In one embodiment of the tablet according to the invention, the kernel further comprises the following excipients:
(i) a filler;
(ii) a disintegrant; and
(iii) a glidant.

In one embodiment:
(i) said filler is selected from MicroceLac® 100 and SMCC90;
(ii) said disintegrant is croscarmellose sodium; and
(iii) said glidant is sodium stearyl fumarate.

In one embodiment:
(i) said filler is MicroceLac® 100;
(ii) said disintegrant is croscarmellose sodium; and
(iii) said glidant is sodium stearyl fumarate.

In one embodiment:
(i) said filler is SMCC90;
(ii) said disintegrant is croscarmellose sodium; and
(iii) said glidant is sodium stearyl fumarate.

In one embodiment:
(i) the weight of said filler represents 58%+1% of the total weight of the kernel;
(ii) the weight of said disintegrant represents 5%+1% of the total weight of the kernel;
(iii) the weight of said glidant represents 4%±1% of the total weight of the kernel; and
(iv) the weight of said compound of Formula I, or a pharmaceutically acceptable salt thereof, represents 33%±1% of the total weight of the kernel.

### Formulation B

In one embodiment of the tablet according to the invention, the kernel further comprises the following excipients:
(i) a first filler;
(ii) a second filler; and
(iii) a lubricant.

In one embodiment:
(i) said first filler is Ludipress®;
(ii) said second filler is microcrystalline cellulose; and
(iii) said lubricant is sodium stearyl fumarate.

In one embodiment:
(i) the weight of said first filler represents 43%±1% of the total weight of the kernel;
(ii) the weight of said second filler represents 20%±1% of the total weight of the kernel;
(iii) the weight of said lubricant represents 4%±1% of the total weight of the kernel;
(iv) the weight of said compound of Formula I, or a pharmaceutically acceptable salt thereof, represents 33%±1% of the total weight of the kernel.

### Continuous Mini-Batch Direct Compression

In one aspect, the present invention provides a continuous process for manufacturing a tablet based on Formulation A or B described herein, comprising:
(i) feeding the API and components (i)-(iii) from four individual screw feeders into a blender;
(ii) blending the mixture of step (i);
(iii) compressing the blend from step (ii) into tablet kernels; and
(iv) spraying a film coating suspension onto the tablet kernels from step (iii).

### Formulation for Roller Compaction

### Formulation C

In one embodiment of the tablet according to the invention, the kernel further comprises the following excipients:
(i) a first filler;
(ii) a second filler;
(iii) a disintegrant;
(iv) a glidant; and
(v) a flow agent.

In one embodiment:
(i) said first filler is microcrystalline cellulose;
(ii) said second filler is lactose monohydrate;
(iii) said disintegrant is croscarmellose sodium;
(iv) said glidant is sodium stearyl fumarate; and
(v) said flow agent is colloidal silicon dioxide.

In one embodiment:
(i) the weight of said first filler represents 33%±1% of the total weight of the kernel;
(ii) the weight of said second filler represents 23%±1% of the total weight of the kernel;
(iii) the weight of said disintegrant represents 5%±1% of the total weight of the kernel;
(iv) the weight of said glidant represents 4%±1% of the total weight of the kernel;
(v) the weight of said flow agent represents 2%±1% of the total weight of the kernel; and
(vi) the weight of said compound of Formula I, or a pharmaceutically acceptable salt thereof, represents 33%±1% of the total weight of the kernel.

### Roller Compaction

In one aspect, the present invention provides a roller compaction process for manufacturing a tablet based on Formulation C described herein, comprising:
(i) blending the API, first filler, second filler, disintegrant, and glidant;
(ii) screening the blend obtained from step (i);
(iii) screening a 1^{st} portion of the lubricant and adding it to the blend from step (ii);
(iv) performing a roller compaction with the blend from step (iii) to form granules;
(v) screening a 2^{nd} portion of the lubricant and adding it to the granules from step (iv);
(vi) compressing the blend from step (v) into tablet kernels; and
(vii) spraying a film coating suspension onto the tablet kernels from step (vi).

### Uses

In one aspect, the present invention provides a tablet as described herein, for use as a medicament.

In one aspect, the present invention provides a method for treating or preventing a TAAR1(4) mediated disease in a patient, comprising administering one or more tablets described herein to the patient.

In one aspect, the present invention provides a tablet described herein, for use in a method of treating or preventing a TAAR1(4) mediated disease in a patient.

In one aspect, the present invention provides the use of a tablet described herein in a method of treating or preventing a TAAR1(4) mediated disease in a patient.

In one embodiment, said TAAR1(4) mediated disease is selected from depression, anxiety disorders, bipolar disorder, attention deficit hyperactivity disorder (ADHD), stress-related disorders, schizophrenia, Parkinson's disease, Alzheimer's disease, epilepsy, migraine, hypertension, substance abuse, addiction, eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders.

In a preferred embodiment, said TAAR1(4) mediated disease is selected from schizophrenia, substance abuse, and addiction.

In a particularly preferred embodiment, said TAAR1(4) mediated disease is schizophrenia.

In a particularly preferred embodiment, said TAAR1(4) mediated disease is substance abuse.

In a particularly preferred embodiment, said TAAR1(4) mediated disease is addiction.

### EXAMPLES

The following examples are provided for illustration of the invention. They should not be considered as limiting the scope of the invention, but merely as being representative thereof.

### Example 1—Tablet Formation from a Continuous Mini-Batch Direct Compression Process

### Equipment

| Process Step | Class | Subclass | Equipment |
|---|---|---|---|
| Feeding | Screw feeders | — | ThreeTec ZD 12 |
| | | | (“small feeders”) and |
| | | | 22 FB (“large feeder”) |
| Blending | High shear blender | — | Loedige ploughshare |
| | | | mixer 5 L |
| Tablet | Tablet Press | Power Assisted | Korsch XL100 |
| Compression | | | Wipcon |
| Film-Coating | Pan Coating | Perforated Coating | GMPC I |
| | | System | |

### Process

1. Feeding of excipient (i) from the large feeder; and API, excipient (ii), and excipient (iii) from the three small feeders into the mini-batch blender.
2. Blending of the mini-batch in the mini-batch blender.
3. Discharge into the tablet press the mini-batch prepared in steps 1 and 2.
4. Press tablet cores. Perform IPC on the individual tablet weight, hardness, thickness, friability and disintegration time for tablet cores.
5. Repeat steps 1 to 4 as needed to manufacture the desired final batch.
6. Prepare film coating suspension and spray film coat onto tablet cores obtained from step 4. Perform IPC on the average weight, thickness and disintegration time of film-coated tablets.

In one embodiment, the API is ralmitaront mono hydrochloride, excipient (i) is a filler selected from MicroceLac® 100 and SMCC90; excipient (ii) is a disintegrant being croscarmellose sodium; and excipient (iii) is a lubricant being sodium stearyl fumarate (see Examples 5, 7 and 9).

In one embodiment, the API is ralmitaront mono hydrochloride, excipient (i) is a first filler being Ludipress®; excipient (ii) is a second filler being microcrystalline cellulose; and excipient (iii) is a lubricant being sodium stearyl fumarate (see Example 6).

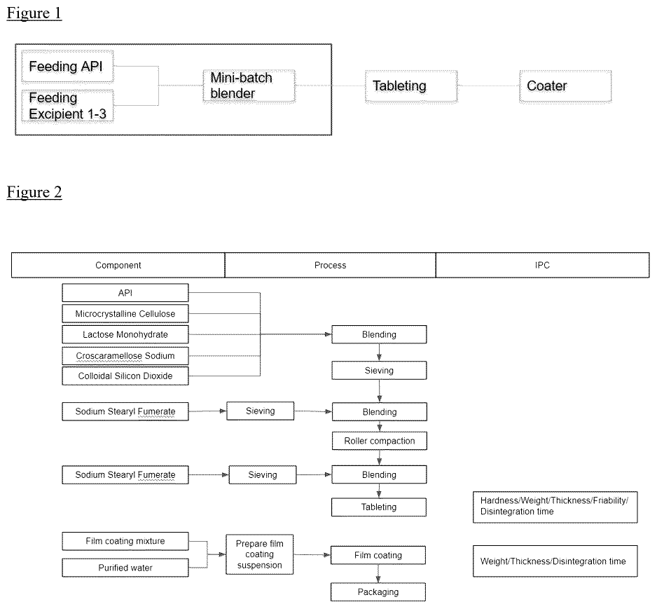

A schematic overview of this process is provided in FIG. 1.

### Example 2—Tablet Formation from a Roller Compaction Process

### Equipment

| Process Step | Class | Subclass | Equipment |
|---|---|---|---|
| Blending | Diffusion mixer | Bin Blender | Servolift Blender |
| Granulation | Dry Granulator | Roller Compaction | Gerteis Minipactor |
| Tablet | Tablet Press | Power Assisted | Korsch XL100 |
| Compression | | | Wipcon |
| Film-Coating | Pan Coating | Perforated Coating | Glatt GC750 |
| | | System | |

### Process

1. Weigh API, Microcrystalline Cellulose, Lactose Monohydrate, Croscarmellose and Colloidal Silicon Dioxide and blend.
2. Screen the obtained blend from step 1.
3. Screen 50% of total amount of Sodium Stearyl Fumarate, add it to the powder blend from step 2 and blend.
4. Perform a roller compaction with the blend.
5. Screen remaining 50% of total amount of Sodium Stearyl Fumarate, add it to the granule from step 4 and blend.
6. Perform tablet manufacturing with final blend obtained from step 5. Perform IPC on the individual tablet weight, hardness, thickness, friability and disintegration time for tablet cores.
7. Prepare film coating suspension and spray film coat onto tablet cores obtained from step 6. Perform IPC on the average weight, thickness and disintegration time of film-coated tablets.
A schematic overview of this process is provided in FIG. 2.

### Example 3—Comparison of Different Fillers for Continuous Mini-Batch Direct Compression

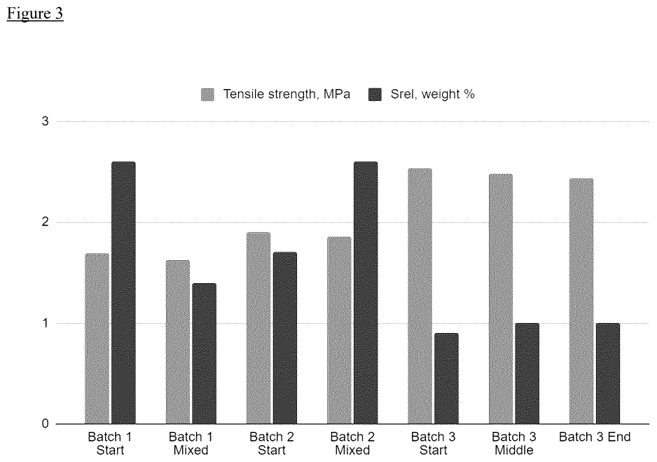

Comparison of the three formulations of examples 5, 6 and 7 surprisingly showed that a blend comprising the filler SMCC90 (Example 7) afforded stronger tablets than comparable blends comprising MicroceLac® 100 (Example 5) or Ludipress® (Example 6). In addition, the standard relative deviation of main compression force (“Srel”) during the tablet compression was lower when using SMCC90 in the blend, compared to when using MicroceLac® 100 or Ludipress®. In other words, the blend described in Example 7, comprising SMCC90 as a filler, yields tablets of higher quality, i.e. stronger tablets, and makes the tabletting process more stable, i.e., reduces the Srel, compared to the blends described in Examples 5 and 6. This is illustrated in FIG. 3.

### Example 4—150 mg Tablet Formulation

| Component | Function | Amount (mg/tablet) | |
|---|---|---|---|
| Ralmitaront mono | Active | 167.40 | mg |
| hydrochloride | Ingredient | (equivalent to | |
| | | 150 | mg of |
| | | the free base) | |
| Microcrystalline Cellulose | Filler | 163.25 | mg |
| Lactose Monohydrate | Filler | 114.35 | mg |
| Croscarmellose Sodium | Disintegrant | 25.00 | mg |
| Sodium Stearyl Fumarate | Glidant | 20.00 | mg |
| Colloidal Silicon Dioxide | Flow Agent | 10.00 | mg |
| Opadry II White | Coating | 20.00 | mg |
| Total Tablet Weight | | 520.00 | mg |

All excipients used in the formulation are compendial (Ph. Eur. and/or USP/NF) grade.

The tablets may be manufactured according to the process described in Example 2.

### Example 5—Alternative 150 mg Tablet Formulation

| Component | Function | Amount (mg/tablet) | |
|---|---|---|---|
| Ralmitaront mono | Active | 167.40 | mg |
| hydrochloride | Ingredient | (equivalent to | |
| | | 150 | mg of |
| | | the free base) | |
| MicroceLac ® 100^{a)} | Filler | 287.6 | mg |
| Croscarmellose Sodium | Disintegrant | 25.00 | mg |
| Sodium Stearyl Fumarate | Lubricant | 20.00 | mg |
| Opadry II White | Coating | 20.00 | mg |
| Total Tablet Weight | | 520.00 | mg |

^{a)}MicroceLac ® 100 is a commercially available excipient consisting of co-processed microcrystalline cellulose and alpha-lactose monohydrate.

All excipients used in the formulation are compendial (Ph. Eur. and/or USP/NF) grade.

The tablets may be manufactured according to the continuous process described in Example 1.

### Example 6—Alternative 150 mg Tablet Formulation

| Component | Function | Amount (mg/tablet) | |
|---|---|---|---|
| Ralmitaront mono | Active | 167.40 | mg |
| hydrochloride | Ingredient | (equivalent to | |
| | | 150 | mg of |
| | | the free base) | |
| Ludipress ® ^{a)} | Filler | 212.6 | mg |
| Microcrystalline cellulose | Filler | 100.0 | mg |
| Sodium Stearyl Fumarate | Lubricant | 20.00 | mg |
| Opadry II White | Coating | 20.00 | mg |
| Total Tablet Weight | | 520.00 | mg |

^{a)} Ludipress ® is a commercially available excipient consisting of co-processed lactose monohydrate, povidone and crospovidone.

All excipients used in the formulation are compendial (Ph. Eur. and/or USP/NF) grade.

The tablets may be manufactured according to the continuous process described in Example 1.

### Example 7—Alternative 150 mg Tablet Formulation

| Component | Function | Amount (mg/tablet) | |
|---|---|---|---|
| Ralmitaront mono | Active | 167.40 | mg |
| hydrochloride | Ingredient | (equivalent to | |
| | | 150 | mg of |
| | | the free base) | |
| SMCC90^{a)} | Filler | 287.6 | mg |
| Croscarmellose Sodium | Disintegrant | 25.00 | mg |
| Sodium Stearyl Fumarate | Lubricant | 20.00 | mg |
| Opadry II White | Coating | 20.00 | mg |
| Total Tablet Weight | | 520.00 | mg |

^{a)}SMCC90 is a commercially available excipient consisting of silicified microcrystalline cellulose.

All excipients used in the formulation are compendial (Ph. Eur. and/or USP/NF) grade. Example 1.

### Example 8—45 mg Tablet Formulation

| Component | Function | Amount (mg/tablet) | |
|---|---|---|---|
| Ralmitaront mono | Active | 50.22 | mg |
| hydrochloride | Ingredient | (equivalent to | |
| | | 45 | mg of |
| | | the free base) | |
| Microcrystalline Cellulose | Filler | 48.98 | mg |
| Lactose Monohydrate | Filler | 34.30 | mg |
| Croscarmellose Sodium | Disintegrant | 7.50 | mg |
| Sodium Stearyl Fumarate | Glidant | 6.00 | mg |
| Colloidal Silicon Dioxide | Flow Agent | 3.00 | mg |
| Opadry II White | Coating | 6.00 | mg |
| Total Tablet Weight | | 156.00 | mg |

All excipients used in the formulation are compendial (Ph. Eur. and/or USP/NF) grade.

The tablets may be manufactured according to the process described in Example 2.

### Example 9—Alternative 45 mg Tablet Formulation

| Component | Function | Amount (mg/tablet) | |
|---|---|---|---|
| Ralmitaront mono | Active | 50.22 | mg |
| hydrochloride | Ingredient | (equivalent to | |
| | | 45 | mg of |
| | | the free base) | |
| MicroceLac ® 100^{a} | Filler | 86.28 | mg |
| Croscarmellose Sodium | Disintegrant | 7.50 | mg |
| Sodium Stearyl Fumarate | Glidant | 6.00 | mg |
| Opadry II White | Coating | 6.00 | mg |
| Total Tablet Weight | | 156.00 | mg |

^{a}MicroceLac ® 100 is a commercially available excipient consisting of co-processed microcrystalline cellulose and alpha-lactose monohydrate.

All excipients used in the formulation are compendial (Ph. Eur. and/or USP/NF) grade. Example 1.

### Example 10—Alternative 45 mg Tablet Formulation

| Component | Function | Amount (mg/tablet) | |
|---|---|---|---|
| Ralmitaront mono | Active | 50.22 | mg |
| hydrochloride | Ingredient | (equivalent to | |
| | | 45 | mg of |
| | | the free base) | |
| Ludipress ®^{a)} | Filler | 63.78 | mg |
| Microcrystalline cellulose | Filler | 30.0 | mg |
| Sodium Stearyl Fumarate | Lubricant | 6.00 | mg |
| Opadry II White | Coating | 6.00 | mg |
| Total Tablet Weight | | 156.00 | mg |

^{a)} Ludipress ® is a commercially available excipient consisting of co-processed lactose monohydrate, povidone and crospovidone.

All excipients used in the formulation are compendial (Ph. Eur. and/or USP/NF) grade. Example 1.

### Example 11—Alternative 45 mg Tablet Formulation

| Component | Function | Amount (mg/tablet) | |
|---|---|---|---|
| Ralmitaront mono | Active | 50.22 | mg |
| hydrochloride | Ingredient | (equivalent to | |
| | | 45 | mg of |
| | | the free base) | |
| SMCC90 | Filler | 86.28 | mg |
| Croscarmellose Sodium | Disintegrant | 7.50 | mg |
| Sodium Stearyl Fumarate | Glidant | 6.00 | mg |
| Opadry II White | Coating | 6.00 | mg |
| Total Tablet Weight | | 156.00 | mg |

^{a)} SMCC90 is a commercially available excipient consisting of silicified microcrystalline cellulose.

All excipients used in the formulation are compendial (Ph. Eur. and/or USP/NF) grade.

The tablets may be manufactured according to the continuous process described in Example 1.

1. A tablet comprising:
(i) a kernel; and
(ii) a coating;
wherein the kernel comprises 5-ethyl-4-methyl-N-[4-[(2S) morpholin-2-yl]phenyl]-1H-pyrazole-3-carboxamide (a compound of Formula I), or a pharmaceutically acceptable salt thereof, 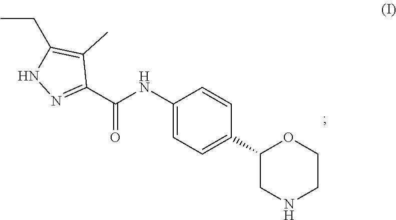 [SMILES: CCc1[nH]nc(C(=O)Nc2ccc([C@H]3CNCCO3)cc2)c1C]
and wherein the kernel further comprises the following excipients:
(i) a first filler;
(ii) a second filler;
(iii) a disintegrant;
(iv) a glidant; and
(v) a flow agent.

2. The tablet according to claim 1, wherein:
(i) said first filler is microcrystalline cellulose;
(ii) said second filler is lactose monohydrate;
(iii) said disintegrant is croscarmellose sodium;
(iv) said glidant is sodium stearyl fumarate; and
(v) said flow agent is colloidal silicon dioxide.

3. The tablet according to claim 1, wherein:
(i) the weight of said first filler represents 33%±1% of the total weight of the kernel;
(ii) the weight of said second filler represents 23%±1% of the total weight of the kernel;
(iii) the weight of said disintegrant represents 5%±1% of the total weight of the kernel;
(iv) the weight of said glidant represents 4%±1% of the total weight of the kernel;
(v) the weight of said flow agent represents 2%±1% of the total weight of the kernel; and
(vi) the weight of said compound of Formula I, or a pharmaceutically acceptable salt thereof, represents 33%±1% of the total weight of the kernel.

4. A tablet comprising:
(i) a kernel; and
(ii) a coating;
wherein the kernel comprises 5-ethyl-4-methyl-N-[4-[(2S) morpholin-2-yl]phenyl]-1H-pyrazole-3-carboxamide (a compound of Formula I), or a pharmaceutically acceptable salt thereof, 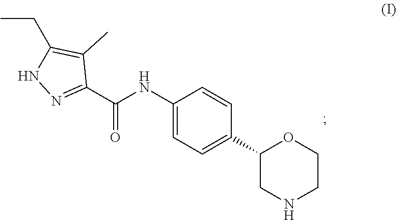 [SMILES: CCc1[nH]nc(C(=O)Nc2ccc([C@H]3CNCCO3)cc2)c1C]
and wherein the kernel further comprises the following excipients:
(i) a filler;
(ii) a disintegrant; and
(iii) a glidant.

5. The tablet according to claim 4, wherein:
(i) said filler is selected from MicroceLac® 100 and SMCC90;
(ii) said disintegrant is croscarmellose sodium; and
(iii) said glidant is sodium stearyl fumarate.

6. The tablet according to claim 4, wherein:
(i) the weight of said filler represents 58%±1% of the total weight of the kernel;
(ii) the weight of said disintegrant represents 5%±1% of the total weight of the kernel;
(iii) the weight of said glidant represents 4%±1% of the total weight of the kernel; and
(iv) the weight of said compound of Formula I, or a pharmaceutically acceptable salt thereof, represents 33%±1% of the total weight of the kernel.

7. A tablet comprising:
(i) a kernel; and
(ii) a coating;
wherein the kernel comprises 5-ethyl-4-methyl-N-[4-[(2S) morpholin-2-yl]phenyl]-1H-pyrazole-3-carboxamide (a compound of Formula I), or a pharmaceutically acceptable salt thereof, 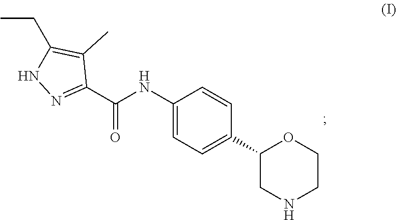 [SMILES: CCc1[nH]nc(C(=O)Nc2ccc([C@H]3CNCCO3)cc2)c1C]
and wherein the kernel further comprises the following excipients:
(i) a first filler;
(ii) a second filler; and
(iii) a lubricant.

8. The tablet according to claim 7, wherein:
(i) said first filler is Ludipress®;
(ii) said second filler is microcrystalline cellulose; and
(iii) said lubricant is sodium stearyl fumarate.

9. The tablet according to claim 7, wherein:
(i) the weight of said first filler represents 43%±1% of the total weight of the kernel;
(ii) the weight of said second filler represents 20%±1% of the total weight of the kernel;
(iii) the weight of said lubricant represents 4%±1% of the total weight of the kernel;
(iv) the weight of said compound of Formula I, or a pharmaceutically acceptable salt thereof, represents 33%±1% of the total weight of the kernel.

10. The tablet according to claim 1, comprising the compound of Formula I in the form of its mono hydrochloric acid salt.

11. The tablet according to claim 1, wherein the dose strength is 40 mg to 160 mg.

12. A process for manufacturing a tablet according to claim 1, comprising:
(i) blending the compound of Formula I, first filler, second filler, disintegrant, and glidant;
(ii) screening the blend obtained from step (i);
(iii) screening a first portion of the lubricant and adding it to the blend from step (ii);
(iv) performing a roller compaction with the blend from step (iii) to form granules;
(v) screening a second portion of the lubricant and adding it to the granules from step (iv);
(vi) compressing the blend from step (v) into a tablet kernel; and
(vii) spraying a film coating suspension onto the tablet kernel from step (vi).

13. A process for manufacturing a tablet according to claim 4, comprising:
(i) feeding the API and components (i)-(iii) from four individual screw feeders into a blender;
(ii) blending the mixture of step (i);
(iii) compressing the blend from step (ii) into tablet kernel; and
(iv) spraying a film coating suspension onto the tablet kernel from step (iii).

14. The tablet according to claim 1, wherein the coating comprises:
(i) HPMC;
(ii) lactose monohydrate;
(iii) titanium dioxide; and
(iv) macrogol.

15. The tablet according to claim 14, wherein:
(i) HPMC represents 34%±1% of the total weight of the coating;
(ii) lactose monohydrate represents 28%±1% of the total weight of the coating;
(iii) titanium dioxide represents 26%±1% of the total weight of the coating; and
(iv) macrogol represents 12%±1% of the total weight of the coating.

16. (canceled)

17. A method for treating depression, anxiety disorders, bipolar disorder, attention deficit hyperactivity disorder (ADHD), stress-related disorders, schizophrenia, Parkinson's disease, Alzheimer's disease, epilepsy, migraine, hypertension, substance abuse, addiction, eating disorders, diabetes, diabetic complications, obesity, dyslipidemia, disorders of energy consumption and assimilation, disorders and malfunction of body temperature homeostasis, disorders of sleep and circadian rhythm, and/or cardiovascular disorders in a patient, comprising administering one or more tablets according to claim 1, to the patient.

18-20. (canceled)

21. The tablet according to claim 11, wherein the dose strength is 45 mg or 150 mg.

22. The tablet according to claim 4, comprising the compound of Formula I in the form of its mono hydrochloric acid salt.

23. The tablet according to claim 4, wherein the dose strength is 40 mg to 160 mg.

24. The tablet according to claim 23, wherein the dose strength is 45 mg or 150 mg.

25. The tablet according to claim 4, wherein the coating comprises:
(i) HPMC;
(ii) lactose monohydrate;
(iii) titanium dioxide; and
(iv) macrogol.

26. The tablet according to claim 25, wherein:
(i) HPMC represents 34%±1% of the total weight of the coating;
(ii) lactose monohydrate represents 28%±1% of the total weight of the coating;
(iii) titanium dioxide represents 26%±1% of the total weight of the coating; and
(iv) macrogol represents 12%±1% of the total weight of the coating.

27. A method for treating depression, anxiety disorders, bipolar disorder, attention deficit hyperactivity disorder (ADHD), stress-related disorders, schizophrenia, Parkinson's disease, Alzheimer's disease, epilepsy, migraine, hypertension, substance abuse, addiction, eating disorders, diabetes, diabetic complications, obesity, dyslipidemia, disorders of energy consumption and assimilation, disorders and malfunction of body temperature homeostasis, disorders of sleep and circadian rhythm, and/or cardiovascular disorders in a patient, comprising administering one or more tablets according to claim 4 to the patient.

28. The tablet according to claim 7, comprising the compound of Formula I in the form of its mono hydrochloric acid salt.

29. The tablet according to claim 7, wherein the dose strength is 40 mg to 160 mg.

30. The tablet according to claim 29, wherein the dose strength is 45 mg or 150 mg.

31. The tablet according to claim 7, wherein the coating comprises:
(i) HPMC;
(ii) lactose monohydrate;
(iii) titanium dioxide; and
(iv) macrogol.

32. The tablet according to claim 31, wherein:
(i) HPMC represents 34%±1% of the total weight of the coating;
(ii) lactose monohydrate represents 28%±1% of the total weight of the coating;
(iii) titanium dioxide represents 26%±1% of the total weight of the coating; and
(iv) macrogol represents 12%±1% of the total weight of the coating.

33. A process for manufacturing a tablet according to claim 7, comprising:
(i) feeding the compound of Formula I and components (i)-(iii) from four individual screw feeders into a blender;
(ii) blending the mixture of step (i);
(iii) compressing the blend from step (ii) into a tablet kernel; and
(iv) spraying a film coating suspension onto the tablet kernel from step (iii).

34. A method for treating depression, anxiety disorders, bipolar disorder, attention deficit hyperactivity disorder (ADHD), stress-related disorders, schizophrenia, Parkinson's disease, Alzheimer's disease, epilepsy, migraine, hypertension, substance abuse, addiction, eating disorders, diabetes, diabetic complications, obesity, dyslipidemia, disorders of energy consumption and assimilation, disorders and malfunction of body temperature homeostasis, disorders of sleep and circadian rhythm, and/or cardiovascular disorders in a patient, comprising administering one or more tablets according to claim 7 to the patient.
